(19) United States
(12) Patent Application Publication
(10) Pub. No.: US 2025/0302669 A1
(43) Pub. Date: Oct. 2, 2025

(54) Noise reduction goggles

(71) Applicant: Shenzhen Hanmai Technology Co., Ltd, Shenzhen (CN)

(72) Inventor: Wenyuan Zhang, Zhangshu (CN)

(21) Appl. No.: 19/238,385

(22) Filed: Jun. 14, 2025

(30) Foreign Application Priority Data
Mar. 21, 2025 (CN) 202520509885.1

(51) Int. Cl. A61F 9/02; A61F 11/14

(52) U.S. Cl. CPC A61F 9/029; A61F 11/14

(57) ABSTRACT

Noise reduction goggles are provided, which relate to the technical field of protective equipment. The noise reduction goggles include a goggles frame and goggles legs; noise reduction pendants are mounted at the goggles legs; each noise reduction pendant is provided with an adjustment mechanism, a cotton thread, and a noise reduction earplug; the cotton thread is connected to the adjustment mechanism and the noise reduction earplug; and the adjustment mechanisms adjust lengths of the cotton threads, to release or withdraw the noise reduction earplugs. In summary, by efficient integration of the goggles and the noise reduction earplugs, one-key return, and automatic positioning, a user experience and operation convenience are improved, and stability and safety of equipment are ensured.

### TECHNICAL FIELD

The present disclosure relates to the technical field of protective equipment, and in particular, to noise reduction goggles.

### BACKGROUND

In many specific activity venues and working environments, such as a shooting sport field, a cycling sport track, a logging working area, and a decoration working site, there are diverse and strict protection needs for users.

From the perspective of eye protection, there are flying debris in these scenes, such as debris jumping out of a gun barrel during shooting, flying stones during cycling, falling wood chips during logging, and small particles raised during decoration, which can easily cause damage to eyes. Meanwhile, under long-time exposure to strong light, especially ultraviolet rays and dazzling light sources, eye discomfort may be caused, and the eyesight may be even affected. Therefore, professional goggles have become necessary equipment. The goggles only need to have a physical protection performance to block foreign object impact, but also should have good anti-ultraviolet and anti-glare functions, to comprehensively ensure the safety of the eyes.

In terms of ear protection, these places generally have loud noise. The roar of a firearm during shooting, the whistling wind during cycling, the operating sound of logging machinery, the knocking sound of a decoration tool, and the like may cause hearing loss if ears are exposed to such high decibel environments for a long time. Wearing earplugs has become a common measure for effectively blocking noise and achieving physical noise reduction.

However, in actual use, existing protective equipment has many problems. Traditional separate earplugs are inconvenient to store and easily lost when a user takes off the earplugs for talking. This undoubtedly increases the use costs. Some earplugs that are fixedly connected by using connecting ropes or pull ropes solve the problem of dropping. However, during use, a rope body swinging behind the ears is not aesthetically pleasing and poses a safety hazard. For example, the rope body may block another object and injure a user.

The patent document No. CN201173999Y discloses multifunctional specialized shooting glasses, and the patent document No. CN213310869U discloses protective glasses for wood processing, both of which have improvements on goggles, but still have the above problems.

Therefore, the current protective equipment in these specific scenes, whether it is eye protection goggles or ear protection earplugs, has defects in different extents. There is an urgent need for a brand-new and comprehensive protective product to solve these problems, so as to enhance a protective effect on users and user experience in complex environments.

### SUMMARY

In order to overcome the drawbacks described above, the present disclosure aims to provide a technical solution capable of solving the above problems.

The present disclosure provides noise reduction goggles, including a goggles frame and goggles legs; the goggles legs are respectively hinged to two ends of the goggles frame; eye-protection lenses are mounted at the goggles frame; noise reduction pendants are mounted at the goggles legs; each noise reduction pendant is provided with an adjustment mechanism, a cotton thread, and a noise reduction earplug; one end of the cotton thread is connected to the adjustment mechanism, and the other end of the cotton thread is connected to the noise reduction earplug; and the adjustment mechanisms are mounted at the goggles legs and adjust lengths of the cotton threads, to release or withdraw the noise reduction earplugs.

Further, an auxiliary nail is further arranged between each cotton thread and each noise reduction earplug; and one end of the auxiliary nail is fixedly connected to the cotton thread, and the other end of the auxiliary nail is fixedly connected to the noise reduction earplug in a replaceable manner.

Further, the noise reduction pendants are detachably fixedly connected to the goggles legs.

Further, a pendant box is further arranged on each goggles leg and extends from one portion of the goggles leg toward two sides by a distance to form a box structure with an accommodating space inside to accommodate the noise reduction pendant; and the noise reduction pendant is detachably fixedly connected to the goggles leg.

Further, each noise reduction pendant is provided with a base and a top cover; the adjustment mechanism is mounted inside the base; the top cover and the base cooperate with each other and are fixedly connected to form an integral component; an extended guide column is arranged inside the base; the adjustment mechanism is provided with a rotating cylinder member; the rotating cylinder member sleeves the extended guide column and forms a rotatable connection; one end of the cotton thread that is connected to the adjustment mechanism is fixedly connected to the rotating cylinder member to wind around the rotating cylinder member.

Further, the adjustment mechanism is further provided with a first elastic member, a stop member, a second elastic member, and a release member; one end of the first elastic member is fixedly connected to the extended guide column, and the other end of the first elastic member is fixedly connected to the rotating cylinder member, thereby driving the rotating cylinder member to rotate around the extended guide column; one end of the stop member is fixedly connected to the rotating cylinder member, and active teeth are arranged on one side, facing the extended guide column, of the other end of the stop member; one end of the release member sleeves the extended guide column, and passive teeth are arranged on one side, facing the stop member, of the other end of the release member; and one end of the second elastic member resists against the base, and the other end of the second elastic member resists against the release member, thereby driving the passive teeth of the release member and the active teeth of the stop member to cooperate with each other to form a ratchet mechanism.

Further, the eye-protection lenses are detachably mounted inside an arc-shaped slot of the goggles frame.

Further, the eye-protection lenses are further provided with nose supports, and the nose supports are detachably mounted on the eye-protection lenses.

Further, pin shafts are respectively arranged at two ends of the goggles frame; a shaft hole is provided in one end of each goggles leg connected to the goggles frame, causing the goggles leg to cooperate with the pin shaft through the shaft hole to form a hinged connection.

Compared with the prior art, the present disclosure has the beneficial effects below:

1. Integrated design: By integrating the goggles and the noise reduction earplugs into the same product, a user does not need to wear the earplugs separately, so that it is convenient and fast, and troubles of carrying and use are reduced.

2. Automatic adjustment: Automatic positioning and one-key return functions of the cotton threads are achieved through the adjustment mechanisms, so that it is convenient for the earplugs to extend and retract. A user can easily withdraw the earplugs by pressing a key gently. This greatly simplifies a use process and improves convenience of use.

3. Stable and safe fixing: Due to the detachable fixing design of the noise reduction pendant and the cooperation between a clamping slot and a clamping strip, it ensures that the earplugs can be safely stored when not in use, and avoids a potential risk caused by loss and improper placement.

4. Appearance beautification: The integrated design improves the overall aesthetic degree, eliminates disorderliness possibly caused by separate earplugs and their connecting ropes, and avoids discomfort and visual interference during use.

5. Multi-scene applicability: The equipment is applicable to various high-noise environments, such as a shooting gallery, a logging operation, and a cycling sport, can meet protection needs of a user in different scenes and support a user to conveniently take off the earplugs for talking, and has a wide application value.

By the above improvements, the patent implements efficient integration of the goggles and the noise reduction earplugs, provides the one-key return and automatic positioning functions, significantly improves a user experience and operation convenience, and ensures stability and safety of the equipment. The noise reduction goggles are applicable to various high-noise scenes, and can improve practicability and beauty of the product while protecting the eyes and hearing of a user.

The additional aspects and advantages of the present disclosure will be partially provided in the following descriptions, some of which will become apparent from the following descriptions, or learned through the practice of the present disclosure.

### BRIEF DESCRIPTION OF THE DRAWINGS

To describe the technical solutions in the embodiments of the present disclosure or in the prior art more clearly, the following briefly introduces the accompanying drawings for describing the embodiments or the related art. Apparently, the accompanying drawings in the following description show merely some embodiments of the present disclosure, and a person of ordinary skill in the art may still derive other drawings from the accompanying drawings without creative efforts.

FIG. 1 is a schematic diagram of an entire structure of the present disclosure;

FIG. 2 is a schematic structural diagram of goggles legs being in a folded state according to the present disclosure;

FIG. 3 is a schematic structural diagram of noise reduction pendants and goggles legs being in a separated state according to the present disclosure;

FIG. 4 is a schematic structural diagram of goggles lenses and a goggles frame being in a separated state according to the present disclosure;

FIG. 5 is a schematic structural diagram of an arc-shaped slot and an undercut slot according to the present disclosure;

FIG. 6 is a schematic structural diagram of parts inside a noise reduction pendant being in a separated state according to the present disclosure; and

FIG. 7 is a schematic sectional view of a noise reduction pendant of the present disclosure.

Reference numerals and names in the drawings are as follows:

10: goggles frame; 11: limiting portion; 12: arc-shaped slot; 13: undercut slot; 14: flexible accessory; 15: hardware nail; 20: eye-protection lens; 21: clamping block; 22: clamping hole; 23: nose support notch; 24: bulge; 25: noise support; 26: recess hole; 27: limiting slot; 30: goggles leg; 31: pendant box; 32: clamping slot; 33: limiting bottom wall; 40: noise reduction pendant; 41: auxiliary nail; 42: noise reduction earplug; 43: top cover; 44: base; 45: clamping strip; 46: extended guide column; 47: wear-resistant sheet; 48: auxiliary sleeve; 50: adjustment mechanism; 51: rotating cylinder member; 52: fixing hole; 53: first elastic member; 54: stop member; 55: active tooth; 56: second elastic member; 57: release member; and 58: passive tooth.

### DETAILED DESCRIPTION OF THE EMBODIMENTS

The technical solutions in the embodiments of the present disclosure are clearly and completely described below. Apparently, the described embodiments are merely some embodiments of the present disclosure, rather than all of the embodiments. All other embodiments obtained by a person of ordinary skill in the art based on the embodiments of the present disclosure without making creative efforts shall fall within the protection scope of the present disclosure.

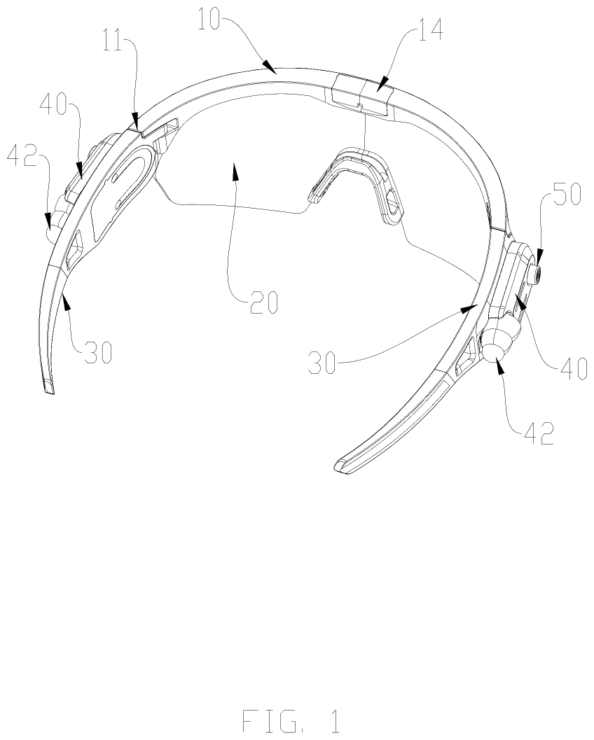

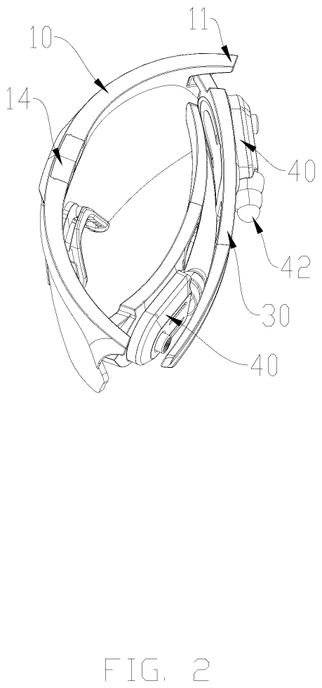

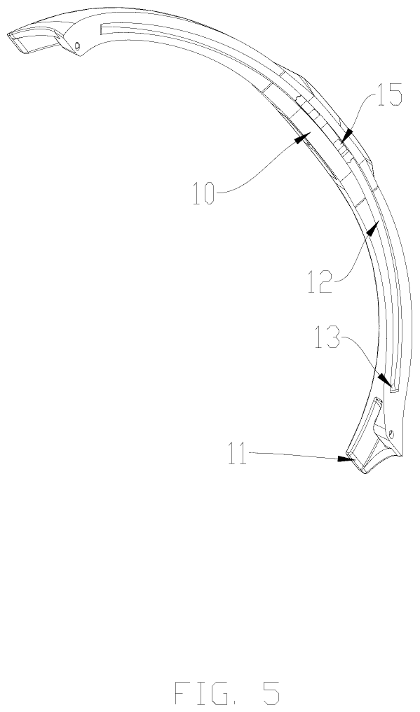

Referring to FIG. 1 to FIG. 7, in an embodiment of the present disclosure, noise reduction goggles include a goggles frame 10 and goggles legs 30. The goggles legs 30 are respectively hinged to two ends of the goggles frame 10. Eye-protection lenses 20 are mounted at the goggles frame 10. Noise reduction pendants 40 are mounted at the goggles legs 30. Each noise reduction pendant 40 is provided with an adjustment mechanism 50, a cotton thread (not shown in the figure), and a noise reduction earplug 42. One end of the cotton thread is connected to the adjustment mechanism 50, and the other end of the cotton thread is connected to the noise reduction earplug 42. The adjustment mechanisms 50 are mounted at the goggles legs 30 and adjust lengths of the cotton threads, to release or withdraw the noise reduction earplugs 42.

Specifically, in shooting sports, cycling sports, logging operation, or decoration operation, it is necessary to protect the eyes of a user to avoid eye damage from flying debris. Meanwhile, there are also anti-ultraviolet and anti-glare requirements. Therefore, professional goggles need to be worn. In these places or scenes, there is still loud noise, so it is necessary to wear earplugs as well, to effectively block the noise and achieve physical noise reduction.

Secondly, people need to talk in a shooting gallery, a sports area, or a working scene, and then people need to take off the earplugs worn and then wear the earplugs again after they finish talking. It is inconvenient to store traditional separate earplugs after the earplugs are taken off, and it is easy to lose the earplugs. This increases the use costs. The earplugs are also fixedly connected by using connecting ropes or pull ropes in the existing art, to prevent the earplugs from dropping. However, during use, it is inconvenient to store the connecting ropes or the pull ropes. It is unattractive because a rope body swings behind the ears, and even the rope body may block another object and cause an injury accident. Therefore, it is necessary for improvement.

Once again, the adjustment mechanisms 50 release the noise reduction earplugs 42, which is equivalent to the adjustment mechanisms 50 releasing the cotton threads, making the cotton threads longer, so that the noise reduction earplugs 42 can be separated from the noise reduction pendants 40 for easy extension and insertion into the ears to achieve a noise reduction effect. The adjustment mechanisms 50 withdraw the noise reduction earplugs 42, which is equivalent to a user taking off the earplugs from the ears, and then the adjustment mechanisms 50 withdraw the cotton threads, making the cotton threads shorter, so that the earplugs are finally clung to or stored on the noise reduction pendants 40, making the product more beautiful and safer.

In addition, in order to facilitate the extension and retraction movement of the cotton threads, auxiliary sleeves 48 can also be arranged inside the noise reduction pendants 40, and the auxiliary sleeves 48 can be fixed between bases 44 and top covers 43, so that the cotton threads can move back and forth through the auxiliary sleeves 48.

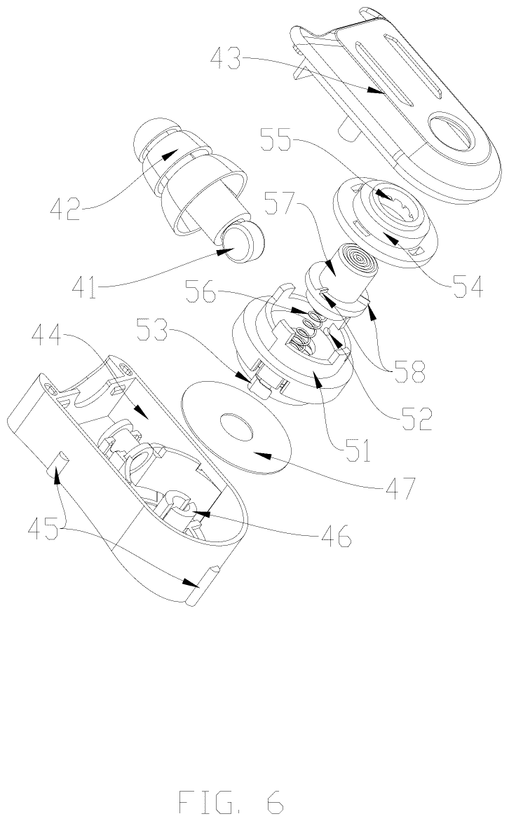

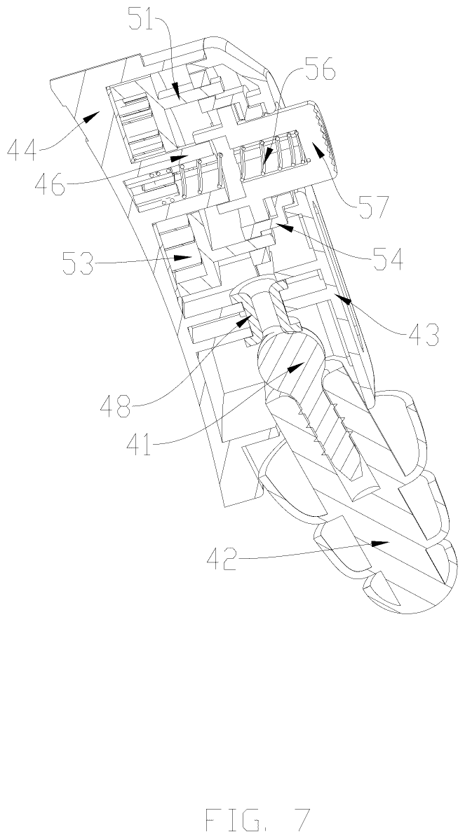

As shown in FIG. 6 to FIG. 7, preferably, an auxiliary nail 41 is further arranged between each cotton thread and each noise reduction earplug 42. One end of the auxiliary nail 41 is fixedly connected to the cotton thread, and the other end of the auxiliary nail 41 is fixedly connected to the noise reduction earplug 42 in a replaceable manner.

Specifically, since the noise reduction earplugs 42 may become dirty and need to be replaced after being used for a period of time, the auxiliary nails 41 can be provided. With spiral structures, the auxiliary nails can be spirally inserted into tail ends of the noise reduction earplugs 42, which can fix the earplugs and be replaced when needed. Each auxiliary nail 41 is preferably made of a plastic material and is provided with a connecting seat at one end for connecting each cotton thread and a threaded tip at the other end, making it easy to spirally insert the auxiliary nail 41 into the tail of the earplug. Each noise reduction earplug 42 is preferably made of a silicone material. Due to the elastic deformation characteristic of the silicone material, noise is effectively isolated. A thread of the auxiliary nail 41 is preferably designed as a coarse thread for manual rotation, and a recess can be reserved at the tail of the earplug, to facilitate the spiral insertion of the auxiliary nail 41.

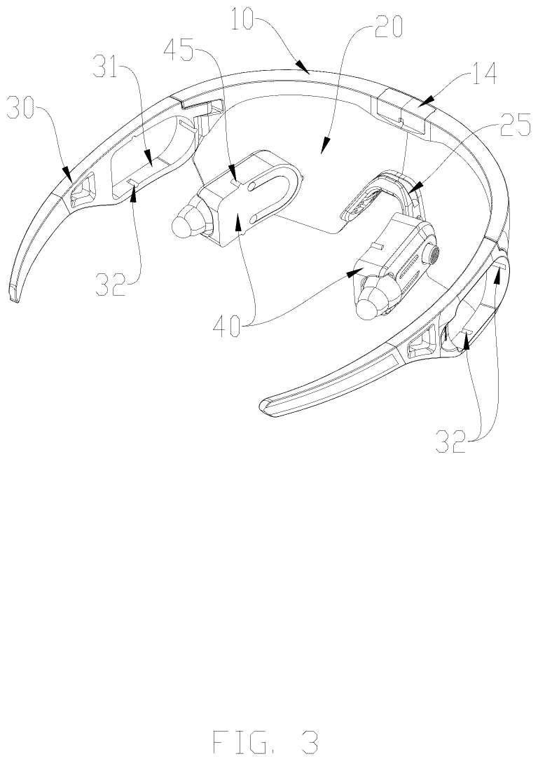

As shown in FIG. 3, preferably, the noise reduction pendants 40 are detachably fixedly connected to the goggles legs 30.

Specifically, shells of the noise reduction pendants 40 can be directly mounted and fixed on the goggles legs 30, or directly integrally formed on the goggles legs 30. In order to facilitate replacement after damage, the noise reduction pendants 40 can also be detachably fixed on the goggles legs 30. For example, bolts and bolt holes are used to cooperate to form the detachable fixed connection, or a snap-in connection achieves the detachable fixed connection, or clamping arms are arranged on the noise reduction pendants 40, so that the noise reduction pendants 40 can be clamped on the goggles legs 30 to form the detachable fixed connection.

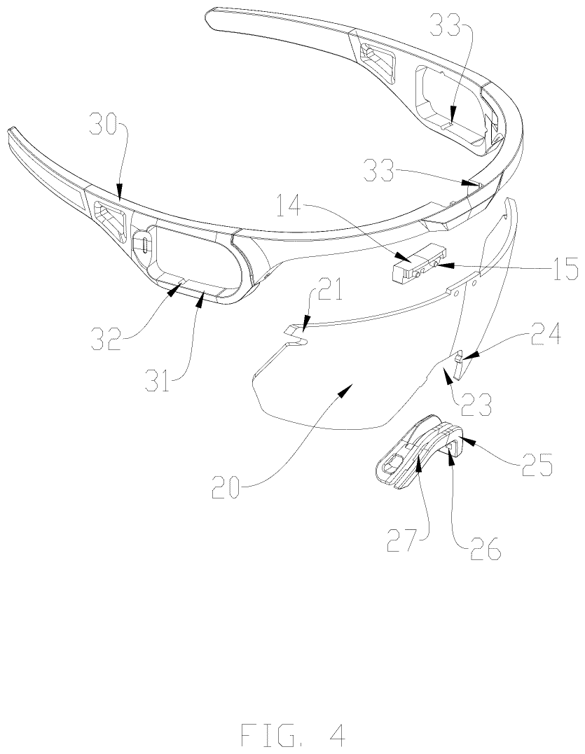

As shown in FIG. 3 to FIG. 4, preferably, a pendant box 31 is further arranged on each goggles leg 30 and extends from one portion of the goggles leg 30 toward two sides by a distance to form a box structure with an accommodating space inside to accommodate the noise reduction pendant 40; and the noise reduction pendant 40 is detachably fixedly connected to the goggles leg 30.

Specifically, a clamping slot 32 is provided in an inner side wall of the pendant box 31, and a clamping strip 45 is arranged at a portion, corresponding to the clamping slot 32, on an outer side wall of the shell of the noise reduction pendant 40. The clamping strip 45 can be embedded into the clamping slot 32 to form an interference fit, thereby forming a detachable and replaceable fixed connection. In order to ensure that the noise reduction pendant 40 can be stably clamped into the pendant box 31, preferably, three groups of clamping slots 32 are respectively provided in inner side walls of three side surfaces of the pendant box 31, and three groups of clamping strips 45 are also respectively arranged on the outer side wall of the shell of the noise reduction pendant 40, so that the three groups of clamping slots 32 and the three groups of clamping strips 45 cooperate with each other to form a stable snap-in connection.

Secondly, in order to prevent the noise reduction pendant 40 from dropping during use, preferably, one end of an embeddings opening of each clamping slot 32 is arranged on one side of the goggles leg 30 facing the head of a user, and a limiting bottom wall 33 is arranged at the other end of the clamping slot 32, so that after the clamping strip 45 is inserted into the clamping slot 32, the clamping strip is restricted at the limiting bottom wall 33 and prevented from dropping out of the other side of the goggles leg 30.

As shown in FIG. 6 to FIG. 7, preferably, each noise reduction pendant 40 is provided with a base 44 and a top cover 43. The adjustment mechanism 50 is mounted inside the base 44. The top cover 43 and the base 44 cooperate with each other and are fixedly connected to form an integral component. An extended guide column 46 is arranged inside the base 44. The adjustment mechanism 50 is provided with a rotating cylinder member 51. The rotating cylinder member 51 sleeves the extended guide column 46 and forms a rotatable connection. One end of the cotton thread that is connected to the adjustment mechanism 50 is fixedly connected to the rotating cylinder member 51 to wind around the rotating cylinder member 51.

Specifically, since one end of the cotton thread is fixed to the rotating cylinder member 51, the rotating cylinder member 51 rotates to wrap the cotton thread around the rotating cylinder member 51. This is equivalent that the rotation of the rotating cylinder member 51 can adjust a length of the cotton thread, thereby driving the cotton thread to release or withdraw the noise reduction earplug 42. In order to rotate the rotating cylinder member 51, a rotating handle (not shown in the figure) can also be provided. One end of the rotating handle is in transmission connection to the rotating cylinder member 51, and the other end of the rotating handle extends out of the top cover 43 for being driven and rotated by a user, thereby adjusting the length of the cotton thread. Alternatively, a side hole (not shown in the figure) can be provided in a side wall of the base 44, so that a portion of an outer circumferential side wall of the rotating cylinder member 51 extends out of the side hole, so that a user moves and rotates the rotating cylinder member 51 at the position of the side hole, thereby adjusting the length of the cotton thread.

Secondly, in order to prevent the cotton thread from being unintentionally stretched, a damping can be applied to the rotation of the rotating cylinder member 51, for example, adding a rubber ring in the existing art between the extended guide column 46 and the rotating cylinder member 51 to achieve a damping effect. Alternatively, a rubber nozzle can be arranged at an outlet corresponding to the cotton thread, and a thread passing hole is provided in the middle of the rubber nozzle. When the cotton thread passes through the thread passing hole, a damping effect can be achieved. The fixed connection between the top cover 43 and the base 44 can be a snap-in connection or a bolt connection.

During use, the noise reduction earplug 42 is pinched with fingers under a low force to deform, thereby applying a gripping force generated by the fingers to the auxiliary nail 41 to prevent earplug damage caused by direct pulling on the noise reduction earplug 42. After the auxiliary nail 41 is stably clamped, a tension is applied in a direction away from the noise reduction pendant 40, to pull the cotton thread out by a length. Then, the noise reduction earplug 42 is pinched and placed into an ear. After expanding, the noise reduction earplug fills the ear to achieve a physical noise reduction effect. When the user needs to listen with the ear, the user pinches the noise reduction earplug 42 and pull the earplug out of the ear. If the earplug is unplugged briefly, the cotton thread does not need to retract. When the noise reduction earplug 42 needs to be used again, the earplug can be quickly inserted back into the ear along the cotton thread. If the noise reduction earplug 42 is not used for a long time, the rotating handle can be turned or the side wall of the rotating cylinder member 51 can be shifted, to drive the rotating cylinder member 51 to rotate synchronously, thus winding the cotton thread to reduce its length for retraction until the noise reduction earplug 42 is clung to or stored on the noise reduction pendant 40, making the overall appearance more beautiful and making the product safer.

As shown in FIG. 6 to FIG. 7, preferably, the adjustment mechanism 50 is further provided with a first elastic member 53, a stop member 54, a second elastic member 56, and a release member 57. One end of the first elastic member 53 is fixedly connected to the extended guide column 46, and the other end of the first elastic member is fixedly connected to the rotating cylinder member 51, thereby driving the rotating cylinder member 51 to rotate around the extended guide column 46. One end of the stop member 54 is fixedly connected to the rotating cylinder member 51, and active teeth 55 are arranged on one side, facing the extended guide column 46, of the other end of the stop member. One end of the release member 57 sleeves the extended guide column 46, and passive teeth 58 are arranged on one side, facing the stop member 54, of the other end of the release member. One end of the second elastic member 56 resists against the base 44, and the other end of the second elastic member resists against the release member 57, thereby driving the passive teeth 58 of the release member 57 and the active teeth 55 of the stop member 54 to cooperate with each other to form a ratchet mechanism.

Specifically, in the previous embodiment, the adjustment on the cotton thread is mainly carried out by manual rotation or shifting, which is not simple and fast enough. Therefore, in this embodiment, a ratchet mechanism can be provided to adjust the cotton thread, thus achieving automatic positioning and one-key return functions. The cotton thread is wound around the outer side wall of the rotating cylinder member 51, and a limiting ring is arranged at one end of the rotating cylinder member 51. A corresponding limiting ring is also formed at the stop member 54 at the other end, thus limiting the winding of the cotton thread and preventing the cotton thread from slipping or deviating during the winding. The rotating cylinder member 51 is usually made of a hard material, so as to cause the cotton thread to be stably wound and unwound.

The first elastic member 53 is preferably configured as a coil spring and mounted inside the rotating cylinder member 51, to provide power for the rotating cylinder member 51 to return. One end of the coil spring is fixed to the extended guide column 46, and the other end is fixed to a side wall of the rotating cylinder member 51. When the rotating cylinder member 51 rotates, the coil spring stores force. After the release member 57 is released, the rotating cylinder member 51 s driven to rotate in an opposite direction to withdraw the cotton thread. The active teeth 55 of the stop member 54 and the passive teeth 58 of the release member 57 cooperate with each other to form the ratchet mechanism for controlling the rotation of the rotating cylinder member 51.

Secondly, the release member 57 sleeves the extended guide column 46 and moves along an axial direction of the extended guide column 46, thus triggering an end surface ratchet for locking or unlocking. During the withdrawal of the cotton thread, a button of the release member 57 is pressed in an axial direction of the rotating cylinder member 51. The button pushes the passive teeth 58 to be separated from the active teeth 55 of the rotating cylinder member 51, to release the ratchet mechanism and release the restriction on the rotation of the rotating cylinder member 51. The elastic force of the coil spring drives the rotating cylinder member 51 to rotate and withdraw the cotton thread. Through the ratchet mechanism, the adjustment mechanism 50 can achieve the automatic positioning and one-key return functions, and is easily and reliably operated.

Furthermore, the active teeth 55 are configured as a plurality of radially arranged ratchet teeth distributed along an end surface of the stop member 54, and the passive teeth 58 are configured as a plurality of radially arranged pawl teeth distributed along an end surface of the release member 57. The ratchet teeth and the pawl teeth are meshed with each other to form the ratchet mechanism. The second elastic member 56 is preferably configured as an ordinary compression spring, an elastic force of which drives the release member 57 to always tend to lock the rotating cylinder member 51 without an external force, thus preventing the rotating cylinder member 51 from returning. To pull the cotton thread, the rotating cylinder member 51 can be driven to rotate, and at the same time, the active teeth 55 on the stop member 54 act as a ratchet and rotate synchronously, while the passive teeth 58 on the release member 57 act as a pawl which can slide over tooth backs of the active teeth 55. When released, the passive teeth 58 may fall into tooth grooves of the active teeth 55 to lock the stop member 54 and the rotating cylinder member 51 and prevent them from rotating counterclockwise, thus achieving automatic positioning of the cotton thread. In an extension direction from the base 44 to the top cover 43, the ratchet teeth tilt counterclockwise and the pawl teeth tilt clockwise, thus ensuring that the pawl teeth can slide over the tooth backs of the ratchet teeth when the cotton thread is pulled. The release member 57 is preferably a button type structure. A user only needs to press the release member 57 to separate the pawl teeth from the ratchet teeth. After the release member 57 is released, the second elastic member 56 (compression spring) drives the release member 57 to be reset.

During use, when the cotton thread needs to be withdrawn, the release member 57 can be pressed to unlock meshed state between the ratchet teeth and the pawl teeth of the ratchet mechanism, thereby releasing the restriction on the rotating cylinder member 51, allowing the coil spring to drive the rotating cylinder member 51 to rotate, and withdrawing the cotton thread. To pull the cotton thread outwards, the ratchet mechanism can achieve a self-locking function. That is, once the release member 57 is released, the compression spring is restored from its deformation, and its elastic force drives the release member 57, so that the ratchet teeth and the pawl teeth of the ratchet mechanism form a new meshed state. That is, the ratchet mechanism may automatically lock the rotating cylinder member 51 again to block the rotation of rotating cylinder member 51 and ensure that the cotton thread will not retract, thereby maintaining a current position of the cotton thread.

In addition, the fixed connection between the stop member 54 and the rotating cylinder member 51 can be achieved by the snap-in connection in the existing art, or the stop member 54 and the rotating cylinder member 51 can be directly fixedly bonded by glue, to form a rotating integrated component. A mounting through hole is provided in a central axis of the rotating cylinder member 51, which facilitates the assembling of the release member 57 and the compression spring. A fixing hole 52 is provided in a top of the rotating cylinder member 51. After the cotton thread passes through the fixing hole 52, the cotton thread can be limited and fixed inside the rotating cylinder member 51 by knotting. In order to make the resetting and extension of the coil spring smoother, a wear-resistant sheet 47 can also be arranged between the coil spring and the base 44, without causing damage to the base 44. In order to press the release member 57, a pressing through hole can also be provided in the top cover 43, so that one end of the release member 57 can protrude out of the pressing through hole, to forming a pressing portion.

As shown in FIG. 1 to FIG. 4, preferably, the eye-protection lenses 20 are detachably mounted inside an arc-shaped slot 12 of the goggles frame 10.

Specifically, in order to facilitate the removal and replacement of the lenses, the arc-shaped slot 12 that is matched with curvatures of the lenses can be provided in one side of the goggles frame 10 facing the downward side, and undercut slots 13 can be provided in two ends of the arc-shaped slot 12, so that clamping blocks 21 at two ends of the lenses can be inserted into the undercut slots 13, to form a detachable fixed connection. It is convenient for removal or replacement by a user after use for a plurality of times.

Secondly, a clamping slot 32 and a flexible accessory 14 are further arranged in the middle of the goggles frame 10, and the flexible accessory 14 is in detachable snap-in connection in the clamping slot 32. Furthermore, by using a process of secondary injection molding on a hardware nail 15, the hardware nail 15 is injection-molded on a relatively soft plastic accessory through the flexible accessory 14, and a clamping hole 22 is provided in a position, corresponding to the hardware nail 15, on each lens. After the lens is mounted into the arc-shaped slot 12, the flexible accessory 14 can be used to be in snap-in connection to the goggles frame 10. Furthermore, under the deformation of the flexible accessory 14, the hardware nail 15 is just inserted into the clamping hole 22, thereby playing an auxiliary fixing role in the mounting of the lens. Meanwhile, the hardware nail 15 is fixed to a flexible portion of the flexible accessory 14 through injection molding, and the clamping hole 22 is located in a non-optical region of the lens, to avoid the impact on the line of sight.

As shown in FIG. 3 to FIG. 4, preferably, the eye-protection lenses 20 are further provided with nose supports 25, and the nose supports 25 are detachably mounted on the eye-protection lenses 20.

Specifically, the nose supports 25 can be also arranged at positions, corresponding to the nose wings of a user, on the lenses, to implement auxiliary supporting on wearing of the lenses. After long-term use, the nose supports 25 may deform or become dirty and needs to be replaced. In order to facilitate later replacement, the nose supports 25 can be fixed by bolt connection or snap-in connection. For example, nose support notches 23 are provided in portions, corresponding to the nose supports 25, on the lenses, and bulges 24 are arranged at outwards extended portions of the nose support notches 23. Recess holes 26 are provided in portions, corresponding to the bulges 24, on the nose supports 25, so that when the nose supports 25 are mounted on the nose support notches 23, the bulges 24 are precisely clamped into the recess holes 26 to fix the nose supports 25. Limiting slots 27 can also be provided in the nose supports 25, and edges of the lenses can be precisely clamped into the limiting slots 27, to further enhance the stability of the nose supports 25. The nose supports 25 are produced by using a dual-color injection molding method for soft plastic and hard plastic. One side of each nose support 25 that is connected to each lens is made of hard plastic by injection molding, while a portion that is in contact with the nose wing of a user is made of soft plastic by injection molding. This can strengthen the strength of a mounting structure of the nose support 25, and enhance the user experience.

As shown in FIG. 1 to FIG. 5, preferably, pin shafts are respectively arranged at two ends of the goggles frame 10. A shaft hole is provided in one end of each goggles leg 30 connected to the goggles frame 10, causing the goggles leg 30 to cooperate with the pin shaft through the shaft hole to form a hinged connection.

Specifically, in order to fold or unfold the goggles legs 30, the pin shafts can also be arranged at the two ends of the goggles frame 10 and are inserted into the shaft holes of the goggles legs 30 to achieve the hinged connection of the goggles legs 30, so that the goggles legs 30 can rotate relative to the goggles frame 10. In order to prevent excessive rotation of the goggles legs 30, which is inconvenient to use, limiting portions 11 can also be arranged at the two ends of the goggles frame 10 to limit and block the rotation of the goggles legs 30, so that the goggles legs 30 can be comfortably worn. The limiting portions 11 are extended ends on the goggles frame 10, and cooperate with missing portions of the goggles legs 30 to limit rotation angles of the goggles legs 30 and ensure wearing stability.

For those skilled in the art, it is apparent that the present disclosure is not limited to the details of the exemplary embodiments mentioned above, and can be implemented in other specific forms without departing from the spirit or basic features of the present disclosure. Therefore, in any perspective, the embodiments should be regarded as exemplary and non-restrictive. The scope of the present disclosure is limited by the accompanying claims rather than the above description. Therefore, all changes within the meaning and scope of the equivalent conditions of the claims within the present disclosure.

What is claimed is:

1. Noise reduction goggles, comprising a goggles frame and goggles legs, wherein the goggles legs are respectively hinged to two ends of the goggles frame; eye-protection lenses are mounted at the goggles frame; noise reduction pendants are mounted at the goggles legs; each noise reduction pendant is provided with an adjustment mechanism, a cotton thread, and a noise reduction earplug; one end of the cotton thread is connected to the adjustment mechanism, and the other end of the cotton thread is connected to the noise reduction earplug; and the adjustment mechanisms are mounted at the goggles legs and adjust lengths of the cotton threads, to release or withdraw the noise reduction earplugs.

2. The noise reduction goggles according to claim 1, wherein an auxiliary nail is further arranged between each cotton thread and each noise reduction earplug; and one end of the auxiliary nail is fixedly connected to the cotton thread, and the other end of the auxiliary nail is fixedly connected to the noise reduction earplug in a replaceable manner.

3. The noise reduction goggles according to claim 1, wherein the noise reduction pendants are detachably fixedly connected to the goggles legs.

4. The noise reduction goggles according to claim 3, wherein a pendant box is further arranged on each goggles leg and extends from one portion of the goggles leg toward two sides by a distance to form a box structure with an accommodating space inside to accommodate the noise reduction pendant; and the noise reduction pendant is detachably fixedly connected to the goggles leg.

5. The noise reduction goggles according to claim 1, wherein each noise reduction pendant is provided with a base and a top cover; the adjustment mechanism is mounted inside the base; the top cover and the base cooperate with each other and are fixedly connected to form an integral component; an extended guide column is arranged inside the base; the adjustment mechanism is provided with a rotating cylinder member; the rotating cylinder member sleeves the extended guide column and forms a rotatable connection; one end of the cotton thread that is connected to the adjustment mechanism is fixedly connected to the rotating cylinder member to wind around the rotating cylinder member.

6. The noise reduction goggles according to claim 5, wherein the adjustment mechanism is further provided with a first elastic member, a stop member, a second elastic member, and a release member; one end of the first elastic member is fixedly connected to the extended guide column, and the other end of the first elastic member is fixedly connected to the rotating cylinder member, thereby driving the rotating cylinder member to rotate around the extended guide column; one end of the stop member is fixedly connected to the rotating cylinder member, and active teeth are arranged on one side, facing the extended guide column, of the other end of the stop member; one end of the release member sleeves the extended guide column, and passive teeth are arranged on one side, facing the stop member, of the other end of the release member; and one end of the second elastic member resists against the base, and the other end of the second elastic member resists against the release member, thereby driving the passive teeth of the release member and the active teeth of the stop member to cooperate with each other to form a ratchet mechanism.

7. The noise reduction goggles according to claim 1, wherein the eye-protection lenses are detachably mounted inside an arc-shaped slot of the goggles frame.

8. The noise reduction goggles according to claim 1, wherein the eye-protection lenses are further provided with nose supports, and the nose supports are detachably mounted on the eye-protection lenses.

9. The noise reduction goggles according to claim 1, wherein pin shafts are respectively arranged at two ends of the goggles frame; a shaft hole is provided in one end of each goggles leg connected to the goggles frame, causing the goggles leg to cooperate with the pin shaft through the shaft hole to form a hinged connection.
